(19) United States
(12) Patent Application Publication
(10) Pub. No.: US 2013/0051178 A1
(43) Pub. Date: Feb. 28, 2013

(54) RESONANTLY AMPLIFIED SHEAR WAVES

(71) Applicant: Andrey Rybyanets, Rostov on Don (RU)

(72) Inventor: Andrey Rybyanets, Rostov on Don (RU)

(73) Assignee: WAVOMED LTD., Ramat Gan (IL)

(21) Appl. No.: 13/695,847

(22) PCT Filed: Apr. 30, 2011
(86) PCT No.: PCT/IB11/51917

§ 371 Date: Nov. 2, 2012

Related U.S. Application Data
(60) Provisional application No. 61/330,449, filed on May 3, 2010.

(51) Int. Cl. H04B 1/02; A61N 7/00; A61B 8/08

(52) U.S. Cl. 367/138

(57) ABSTRACT

Apparatus and methods for generating resonantly amplified ultrasound shear waves in biological tissue. The apparatus comprises a plurality of transducer elements and a controller for controlling the element excitation such as to cyclically generate the pattern of focal regions having associates shear waves and to create resonant amplification of the shear waves. Resonant amplification of the shear waves is obtained when shear waves generated at one focal region are superposed in phase on shear waves synchronously generated at an adjacent focal region. The generation may be done by burst or continuous wave excitation. In some embodiments, the shear waves are supersonic shear waves.

### CROSS REFERENCE TO RELATED APPLICATIONS

This application claims priority from U.S. Provisional Patent Application No. 61/330,449 filed May 3, 2010 and titled “SUPERSONIC SHEAR WAVES”, which is incorporated herein by reference in its entirety.

### TECHNICAL FIELD

Embodiments of the invention relate in general to methods and apparatus for ultrasonic generation of shear waves and in particular for generation and resonant amplification of shear waves in biological tissues.

### BACKGROUND

Acoustic imaging and medical therapy are generally carried out using ultrasound waves, referred to as longitudinal or compression ultrasound waves, in which material in the medium supporting the wave propagation oscillate parallel to the direction of propagation. Acoustic waves in which material in a medium supporting oscillate perpendicular to a direction of propagation of the waves are referred to as transverse, or shear, waves. Shear waves are not supported in fluids, such as gases and liquids, in which forces that bind molecules in the material are very weak and as a result viscosity is very small. Shear waves are supported in living tissue, but because living tissue has relatively low elasticity and high attenuation coefficient the waves are weak and strongly attenuated and do not propagate far from where they are generated.

Shear waves are used to determine characteristics of a tissue in an imaging procedure referred to as Shear Wave Elasticity Imaging (SWEI). In SWEI, shear waves are generated in a tissue region, typically by radiation force produced by conventional longitudinal waves focused to the tissue region. Shear stress generated by the radiation force propagates as shear waves. The region is imaged with conventional longitudinal ultrasound to sense characteristics of the shear waves. The velocity and attenuation rate of the sensed shear waves are used to provide a map of shear modulus and/or shear viscosity as a function of position in the tissue region. The “shear map” is usable to image and detect lesions, such as cancerous lesions, in the region. Generation of radiation force by an acoustic beam is discussed in an article “The Acoustic Radiation Force” by G. R. Torr; Am J Phys. 52(5), May 1984.

U.S. Pat. No. 5,910,731 to Sarvazyan et. al. and US Patent Application No. 2005/0252295 to Fink et al. describe imaging tissue for medical purposes using acoustic shear waves. US Patent Application No 20090099483 to Rybyanets describes a transducer for generating and rotating an intensity pattern of ultrasound comprising a plurality of elements independently excitable to radiate acoustic energy; and a controller that simultaneously excites some of the elements while leaving at least one element dormant and changes which element is dormant to change the intensity pattern. An article entitled “Supersonic Shear Imaging: A new Technique for Soft Tissue Elasticity Mapping” by Bercoff et al, IEEE Transaction on Ultrasonic, Ferroelectrics and Frequency Control, Vol. 51, No 4 April 2004 pp 396-409, describes generating and imaging a shear wave “supersonic boom” to map tissue elasticity. The shear wave supersonic boom is generated by successively focusing an ultrasound beam at increasing depths in a tissue volume so that a focal region of the beam moves to successively increasing depths in the tissue at a speed greater than the speed of acoustic shear waves in the tissue. The radiation force associated with the focal region functions as a source of shear waves that moves with a speed greater than the speed of the shear waves in the tissue to produce the shear wave supersonic boom.

### SUMMARY

An aspect of some embodiments of the invention (referred to hereinafter simply as “embodiments”) relates to providing a method of generating and resonantly amplifying an acoustic shear wave in a region of a material by exciting a cyclically morphing pattern of focal regions of longitudinal ultrasonic wave intensity in the region. In a cyclically morphing pattern of focal regions, focal regions cyclically appear at different locations. A radiation force generated by the longitudinal ultrasound in each focal region contributes to generating the shear wave. A distance between adjacent locations of focal regions in the pattern and a frequency of the acoustic shear wave are determined to provide constructive interference and resonant amplification of the acoustic shear wave at each focal region. In resonant amplification of a shear wave, a radiation force is repeatedly applied in phase with the shear wave to a region of material in which the shear wave propagates, to amplify the shear wave.

According to an aspect of some embodiments, the cyclical rate of change of the focal region pattern and spacing between locations of the focal regions is such that an acoustic focal region in the pattern appears to move with a velocity substantially equal to a speed of propagation of the shear wave to provide resonant shear wave amplification. In an embodiment, a focal region in the pattern moves continuously between the locations. In an embodiment, the focal region is moved “discontinuously” between locations. The focal region is removed from a first location and recreated at a second adjacent location without appearing at intervening locations. A distance “D” between the first and second locations and a time lapse “τ” between movement from one to the other of locations are such that the focal region appears to move between the first and second locations with a speed “D/τ” equal to a speed of propagation of the shear wave.

In some embodiments of the invention, the distance between adjacent locations of focal regions is determined to be substantially equal to an integer multiple of a wavelength of the generated acoustic shear wave. In some embodiments, the distance between adjacent locations of focal regions is determined to be substantially equal to an odd integer multiple of a half wavelength of the generated acoustic shear wave. The distance between adjacent locations of focal regions is also determined to be small enough so that attenuation of the acoustic shear wave over the distance does not prevent acceptable resonant amplification of the shear wave.

In an embodiment, the locations of the focal regions are substantially coplanar. Optionally, the plane of the locations of the focal regions (“focal plane”) is substantially perpendicular to a direction along which the longitudinal ultrasound waves that generate the focal regions are propagated. The shear wave generated by radiation force and amplified as a result of superposition of shear waves from different properly distanced and electrically excited focal zones therefore moves substantially laterally with respect to direction of propagation of the longitudinal waves, in the focal plane.

According to an aspect of some embodiments of the invention, the cyclical rate of change of the pattern and spacing of the locations of the focal regions is such that acoustic focal regions in the pattern appear to move with a velocity greater than the speed of propagation of the shear wave. As a result, an acoustic shear wave generated by the morphing pattern comprises a relatively narrow supersonic shock wave-front in which shear wave intensity is amplified.

According to an aspect of some embodiments of the invention, the pattern of focal regions is rotated to produce a shear wave. Shear waves produced by the rotating pattern are characterized by a wave-front that propagates in a spiral shape away from the pattern.

According to an aspect of some embodiments of the invention, a focal region of a longitudinal ultrasound wave in a material is moved continuously with a velocity substantially equal to a velocity of an acoustic shear wave in a direction substantially perpendicular to a direction of propagation of the longitudinal ultrasound to generate an amplified shear wave.

An aspect of some embodiments of the invention relates to providing a method of generating an acoustic shear wave in a region of a material by generating a stationary pattern of focal regions of longitudinal ultrasonic wave intensity in the region that provides for resonant amplification of shear waves generated at each focal region. In a stationary pattern, all focal regions appear simultaneously, and each time they appear, they appear in a same pattern of locations.

In an embodiment, the focal regions in the stationary pattern are substantially coplanar and the distance between adjacent focal regions is determined to be substantially equal to an integer multiple of the wavelength of shear waves generated by the focal regions. The distance between adjacent focal regions is also determined to be small enough so that attenuation of the acoustic shear wave over the distance is not so large as to prevent acceptable resonant amplification of the shear wave.

Any of various ultrasound transducers and methods may be used to produce a pattern of ultrasound focal regions that generate shear waves, in accordance with an embodiment of the invention. By way of example, an ultrasound transducer used to generate the pattern may be an annular or sectored focusing transducer, or any of various phased array configurations, such as for example a linear, two or three dimensional phased array of piezoelectric elements. In an embodiment, the distance between locations of focal regions in the focal region pattern and the motion of the focal regions are determined by configuration and frequency and/or phase of excitation of an ultrasound transducer and/or elements thereof used to generate the ultrasound pattern of focal regions. In some embodiments, the motion of a focal region in the pattern is provided, at least in part, by physical motion of an ultrasound transducer. Optionally, a desired frequency of the shear wave is determined by repeatedly exciting the pattern in bursts at a burst repetition frequency substantially equal to the desired frequency of the shear wave. In an embodiment, the pattern is generated by exciting the transducer to generate longitudinal ultrasound waves at a plurality of different frequencies, and the frequency of the shear wave is determined by a beat frequency between the frequencies of the longitudinal waves.

In an embodiment, there is provided an apparatus for generating resonantly amplified ultrasound shear waves in biological tissue, comprising a plurality of transducer elements independently excitable to radiate ultrasound energy to generate a pattern of focal regions, each focal region having associated therewith a radiation force that generates a respective shear wave and a controller configured to control the element excitation such as to generate a pattern of focal regions which appear cyclically at different switching positions and to create resonant amplification of shear waves.

In an embodiment, the region of material in which the shear wave is generated is a biological tissue, such as a tissue region of a patient to provide a diagnostic and/or therapeutic and/or cosmetic procedure for the patient. Optionally, the shear wave is used for Shear Wave Elasticity Imaging (SWEI) of the tissue region. According to an aspect of some embodiments of the invention, the shear wave is used to carry out lithotripsy, tissue ablation and lysis of fat cells for cosmetic removal of adipose tissue.

### BRIEF DESCRIPTION OF FIGURES

Non-limiting examples of embodiments of the invention are described below with reference to figures attached hereto and listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIGS. 1A and 1B schematically show perspective views of an acoustic transducer generating a rotating pattern of longitudinal ultrasound focal regions that generates shear waves, in accordance with an embodiment of the invention;

FIG. 2A schematically illustrates resonant amplification of shear waves, in accordance with an embodiment of the invention;

FIG. 2B schematically shows apparatus implementing resonant amplification as shown in FIG. 2A, in accordance with an embodiment of the invention;

FIG. 3A schematically shows an acoustic transducer generating a cyclically morphing acoustic pattern of five focal regions of longitudinal ultrasound intensity that generates shear waves in accordance with an embodiment of the invention;

FIGS. 3B and 3C schematically show the acoustic intensity pattern shown in FIG. 2A at times different from a time at which it is shown in FIG. 2A, in accordance with an embodiment of the invention;

FIG. 4 schematically shows a pattern of focal regions of ultrasound intensity generated by an ultrasound transducer in accordance with an embodiment of the invention.

### DETAILED DESCRIPTION

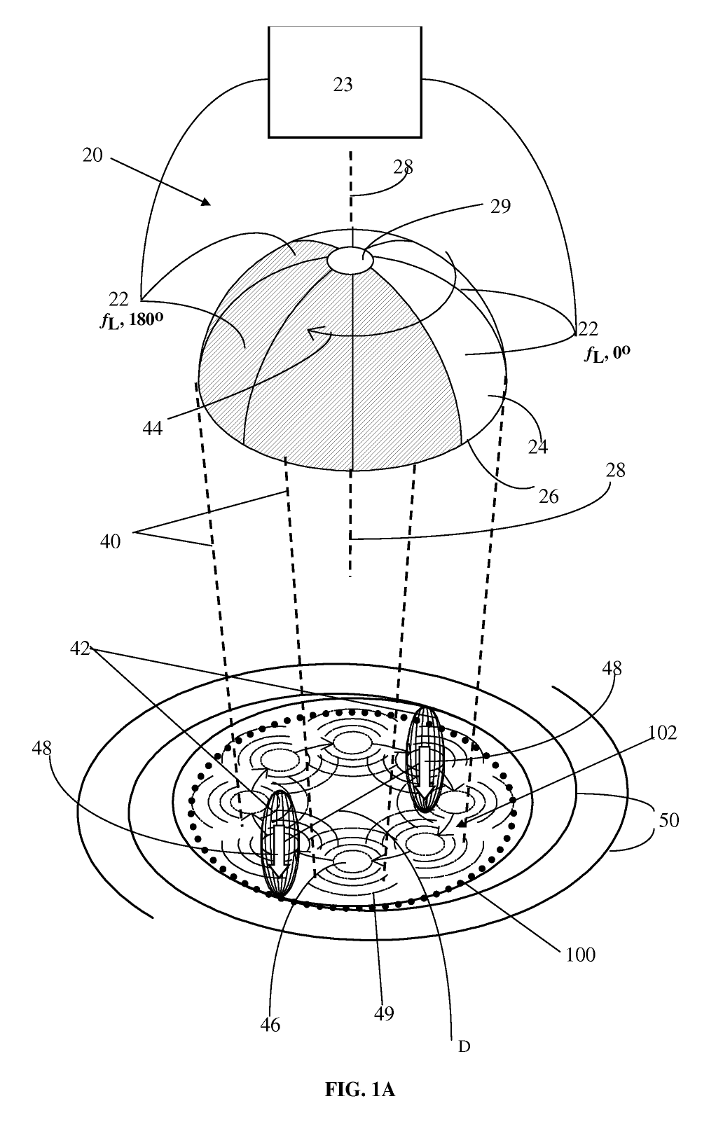

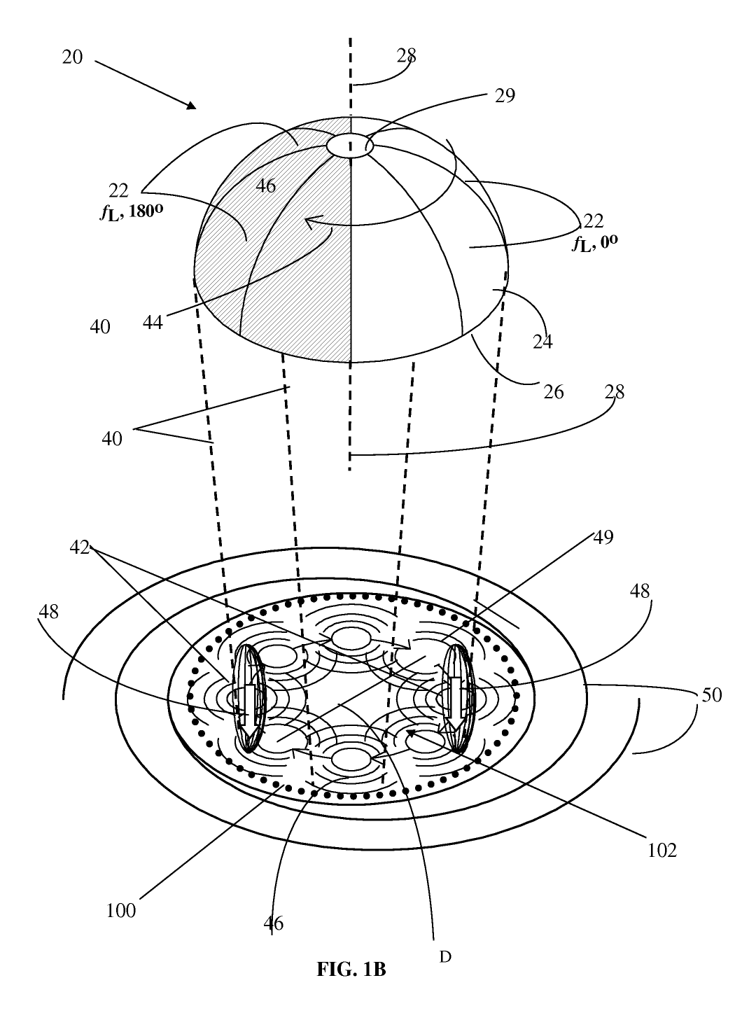

FIGS. 1A and 1B schematically show a perspective view of a multi-element spherical cap acoustic transducer 20 excited to illuminate optionally a tissue region of a patient with longitudinal focused ultrasound and produce a rotating pattern of ultrasound focal regions in the tissue region that generates an amplified shear wave in the region, in accordance with an embodiment of the invention. The tissue region is schematically represented by a dashed circle 100, and will be referred to as “tissue region 100”. Ultrasound radiated by cap transducer 20 to illuminate tissue region 100 is schematically represented by dashed lines 40 extending from the cap transducer to tissue region 100.

Acoustic transducer 20 optionally comprises an even number of identical sectors 22 or “transducer elements” each having an “external” electrode (not shown), referred to as a “sector electrode” on an exterior, convex surface 24 of the transducer that substantially covers the sector area. By way of example, in FIGS. 1A and 1B cap transducer 20 is schematically shown having eight sectors 22. The transducer optionally has a single large “interior” electrode (not shown), referred to as a “common electrode” covering an interior, concave surface 26 of the cap transducer. Cap transducer 20 has an axis 28 and is formed optionally having a hole 29 centered on the axis for convenience of production and to provide a convenient location for a sensor, optionally an acoustic sensor, for monitoring an acoustic field generated by the cap transducer.

The sector electrode of each sector 22 is coupled to a suitable power supply (not shown). A controller 23 is controllable to apply an AC voltage between the sector electrode and the common electrode to excite the sector to generate ultrasound waves independently of excitation of the other sectors. The controller is further configured to control the element excitation such as to generate a pattern of focal regions which appear cyclically at different switching positions and to create resonant amplification of shear waves. Although not shown in other figures, it is to be understood that a controller such as controller 23 is used in every embodiments described herein.

In an embodiment of the invention, the sectors in a same first half of cap transducer 20 are excited by bursts of same frequency, in phase, alternating “excitation” voltage. The frequency of the excitation voltage is represented by “f_{L}” and a repetition frequency of the burst excitation is represented by “v”. Sectors 22 in a second half of the cap transducer are also excited simultaneously with excitation of sectors 22 in the first half of the cap transducer at burst repetition frequency v and frequency f_{L}, but with the phase of the excitation voltage 180° out-of-phase with the excitation voltage of sectors 22 in the first half of the cap transducer.

For convenience of presentation, sectors 22 in the first half, also referred to as the “0° half” of cap transducer 20, are un-shaded and arbitrarily assigned a relative phase of 0°. Some of the sectors in the 0° half of cap transducer 20 are labeled with excitation voltage frequency “f_{L}” and “0°”. Sectors 22 in the second half, also referred to as the “180° half”, of the cap transducer are 180° out-of-phase with the 0° sectors. Some of the sectors in the 180° half are shown shaded and labeled with frequency f_{L} and 180°. Sectors 22 in the 0° and 180° halves are also referred to as 0° and 180° sectors respectively.

The excitation configuration of sectors 22 shown in FIG. 1A generates longitudinal ultrasound waves that illuminate a focal zone 102 in tissue region 100 and are characterized by a pair of localized, high intensity longitudinal ultrasound focal regions, schematically represented by ellipsoids 42 in the focal zone. Focal regions 42 are substantially equidistant from axis 28 of cap transducer 20 and are separated by a distance “D” along a line 44 in focal zone 102 that intersects the axis. Distance D is determined, as discussed below, by the geometry of cap transducer 20 and the frequency f_{L} of longitudinal ultrasound waves transmitted by the cap transducer.

In accordance with an embodiment of the invention, focal regions 42 are rotated about axis 28 at an angular frequency ω by rotating the 0° and 180° halves of cap transducer 20 about axis 28 with an angular velocity ω radians/s and an angular frequency (or rotation frequency) ω/2π. Optionally, 0° and 180° halves are rotated by switching a 180° sector adjacent to the 0° half of cap transducer 20 to excitation at 0° (i.e. in phase with excitation of the 0° sectors) so that it becomes a 0° sector, and switching a 0° sector that is adjacent to the 180° half of cap transducer to excitation at 180° so that it becomes a 180° sector.

To provide rotation at angular frequency ω/2π, switching for a cap transducer comprising N sectors is done at a switching frequency equal to Nω/2π, where N is a number of sectors in the cap transducer. For cap transducer 20 shown in FIG. 1A, N=8 and the switching frequency is 8ω/2π. The rotation direction is schematically represented by a curved arrow 44 and is optionally clockwise as seen from convex side 24 of cap transducer 20. Circles 46 in focal zone 102 indicate positions of focal regions 42 at different sequential “switching times” of excitation voltage. At these switching times, excitation voltage applied to sector electrodes is switched to rotate focal regions about axis 28. Positions indicated by circles 46 are referred to as “switching positions”.

FIG. 1B schematically shows cap transducer 20 shown in FIG. 1A immediately after a first switching time following a time at which cap transducer 20 is shown in FIG. 1A. Relative to shaded (180°)) and un-shaded (0°) halves in FIG. 1A, shaded and un-shaded halves in FIG. 1B are rotated clockwise about axis 28 by an angle equal to an angular extent of a sector 22. Focal regions 42 are also correspondingly rotated clockwise by an angular extent of a sector 22. Longitudinal ultrasound waves 40 radiated by cap transducer 20 to focal zone 102 generate radiation forces at locations of the focal zone on which they are incident. These radiation forces are proportional to amounts of energy from the ultrasound that are absorbed and reflected at the locations. The radiation force at a given location has a direction parallel to the ultrasound incidence direction (the direction of arrows 48).

For biological animal tissue, the radiation force F generated by ultrasound incident on a region of the tissue is generally assumed to be proportional to an amount of energy absorbed from the ultrasound by the region. If the tissue at the location is characterized by an ultrasound absorption coefficient α, and if the intensity of incident ultrasound at the location is “I”, then F can be written as F=2αI/c, where c is the velocity of longitudinal ultrasound waves at the location. I is relatively very large in focal regions 42, and as a result, radiation forces in these regions are also relatively very large. In FIGS. 1A and 1B, these forces are schematically represented by block arrows 48 and each is referred to as “radiation force 48”. However, intensity I and its associated radiation force in a given focal region 42 are of course not constant throughout the focal region, but have a maximum substantially in the center of the focal region and decrease towards edges of the region.

As a focal region 42 is rotated about axis 28, at each switching position 46 of the focal region, radiation force 48 repeatedly displaces tissue in a direction of arrow 48 at a repetition frequency equal to the burst frequency v of excitation of sectors 22. The cyclical strain of tissue in a focal region 42 caused by the changing radiation force generates shear waves having frequency f_{S} equal to the burst frequency v. These shear waves propagate away laterally in the focal plane from the position of the focal region. Shear waves generated at, and propagating away, from each of switching positions 46 when a focal region 42 occupies the switching position are indicated by arcs 49 and referred to as “shear waves 49”.

Shear waves 49 interfere constructively to generate a spiral shaped acoustic shear wave front 50 (“Mach spiral”) which propagates outward from focal zone 102. The pitch of the spiral, or the number of turns of the spiral per unit distance along a given radial direction from a center about which the spiral winds, decreases with the radial distance. That is, the distance between adjacent turns of the spiral increases with the radial distance. The pitch increases with increasing angular velocity ω.

It is noted that for any pair of switching positions 46, focal regions 42 generate shear waves 49 that interfere constructively at the center of tissue region 100. If a period between switching times (i.e. 1/f_{S}), is equal to a multiple of a period of shear waves 49, shear waves generated at all the switching positions constructively interfere at the center of focal region 100 and produce a relatively intense acoustic shear field at the center.

In accordance with an embodiment of the invention, distance D, longitudinal ultrasound wave frequency f_{L}, burst frequency v and angular velocity ω are matched to provide resonant amplification of shears waves 49. Resonant amplification of shear waves 49 occurs if the magnitude of an apparent velocity V_{A} with which focal regions 42 move as they rotate around axis 28 is substantially equal to the magnitude of velocity V_{S} of shear waves 49. V_{A} is substantially equal to ωD/2. Since D is a function of radius of curvature R_{c}, aperture A_{c} and phase difference φ between the two halves of cap transducer 20 as well as wavelength λ_{L} of longitudinal waves radiated by the cap transducer, V_{A} can be written |V_{A}|=ωD(A_{c},R_{c},λ_{L},φ)/2 to explicitly exhibit the dependence of D on transducer geometry and longitudinal wavelength λ_{L}. The V_{S} of shear waves in animal tissue is a function of frequency of the shear waves. Since, as noted above, the frequency f_{S} of shear waves generated by radiation force 48 is equal to burst frequency v, V_{S} may be written |V_{S}(v)| to explicitly show the dependence of the shear velocity on its frequency (dispersion) and thereby on the burst frequency of longitudinal ultrasound waves 40. A condition for resonant amplification of shear waves 49, in accordance with an embodiment of the invention, may therefore be expressed as:

|V_{A}|=ωD(A_{c},R_{c},λ_{L},φ)/2=|V_{S}(v)|

By way of a numerical example, for a phase difference φ=180°, as shown in FIGS. 1A and 1B, assume that cap transducer 20 has an aperture equal to 85 mm, a radius of curvature equal to about 54 mm, and that frequency of excitation voltage applied to sector electrodes of sectors 22 is equal to 1 MHz. Then D is equal to about 1.5 mm (for φ=90° instead of 180°, but otherwise the same values noted above, D=0.75 mm). Assume that burst frequency v is equal to about 10 kHz, then |V_{S}(v)| is equal to about 1.64 m/s. Using the values above, resonant amplification of shear waves 49 occurs for an angular velocity ω that satisfies ω=2186 radians/s. For this angular velocity, the focal regions 42 rotate at about 348 rotations/s.

It is noted that whereas in FIGS. 1A and 1B, focal regions 42 are rotated by rotating excitation of sectors 22, embodiments of the invention are not limited to rotating focal regions of a sectored cap transducer by rotating excitation of sectors in the transducer. For example, same halves of transducer 20 may be excited out-of-phase by 90° or 180° and the cap transducer physically rotated about axis 28 to rotate the focal region. By way of another example, a phased array transducer may be operated to steer focal regions so that they rotate along a circular trajectory.

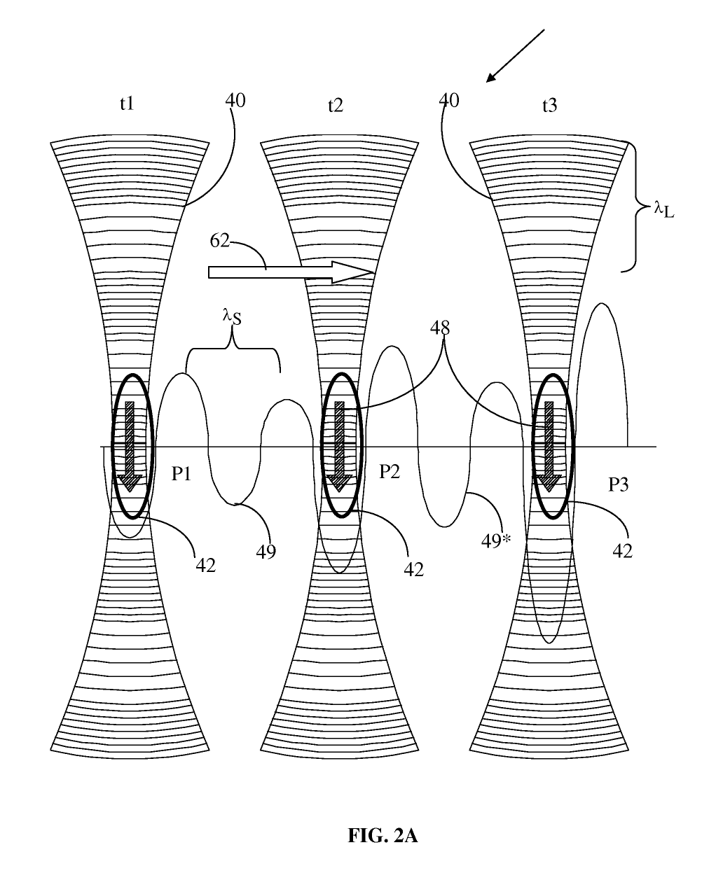

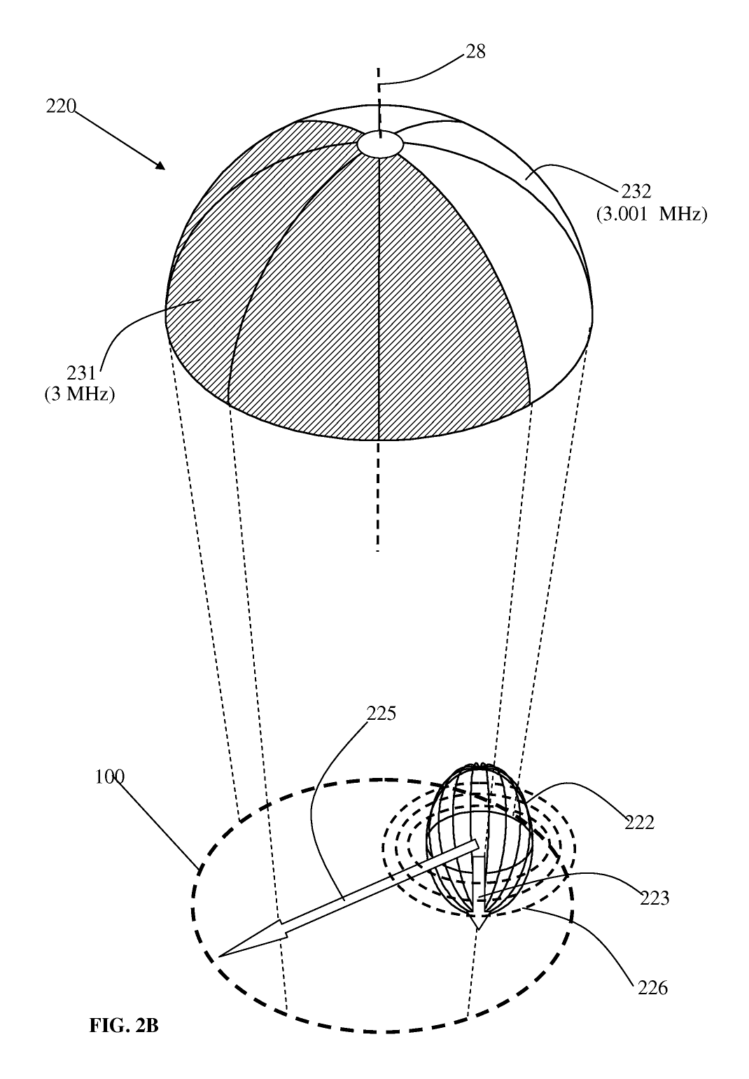

FIG. 2A illustrates schematically, in a highly simplified manner, a process by which the rotation of longitudinal focal regions 42 shown in FIGS. 1A and 1B resonantly amplify shear waves 49. The figure and discussion thereof below are provided as a non-limiting explanation of the resonant amplification provided by rotating focal regions 42. The figure shows schematically the generation of a focal region 42 in focal zone 102 by longitudinal ultrasound waves 40 at sequential switching times t1, t2, and t3, at which excitation voltage to sectors 22 of cap transducer 20 is switched to rotate the focal region around axis 28 of the cap transducer. A portion of the path that the focal region travels as it rotates clockwise (see FIGS. 1A and 1B) around axis 28 is represented by a line 60. At times t1, t2, and t3, focal region is located at switching positions P1, P2 and P3 along its path 60 around axis 28. The hourglass shape of longitudinal ultrasound waves 40 at each position P1, P2 and P3 schematically represent focusing of the ultrasound to produce the focal region at the position.

At time t1 and position P1, radiation force 48 generates an acoustic shear wave 49 which propagates to the right (clockwise in FIGS. 1A and 1B) with a velocity |V_{S}(v)| in a direction indicated by a block arrow 62. As the shear wave propagates to the right it is attenuated as indicated by its decreasing amplitude in the direction of arrow 62. At time t2 the attenuated shear wave reaches position P2. However, focal region 42 travels at a velocity V_{V} having magnitude equal to the magnitude of shear wave velocity V_{S}(v) and therefore it “appears” at position P2 just when attenuated shear wave 49 from P1 reaches P2. The focal region now at position P2 is referred to as a “recreated” focal region 42. As a result, at P2, radiation force 48 generated by recreated focal region 42 is in phase with the attenuated shear wave 49 and generates another shear wave that constructively interferes with and adds to the attenuated shear wave 49 that arrived at P2 from P1. As a result, an amplified shear wave 49* propagates away from P2 towards position P3. The amplified shear wave 49* undergoes attenuation in propagating from P2 to P3 and amplification when it reaches P3 and meets another recreated focal region 48, in a repetition of the attenuation and amplification process of shear wave 49 between positions P1 and P2. The process of attenuation and amplification repeats itself at each new switching position of focal region 42 as the focal region rotates around axis 28 to provide substantial amplification of the original shear wave 49 emitted at switching position P1.

Amplification may be modeled as a geometrical series. Let shear wave 49 generated at switching position P1 have an arbitrary amplitude equal to 1. Let the amplitude of a shear wave that propagates between switching times tn and t(n+1) from position Pn to position P(n+1) be attenuated by a factor “r”. If the shear wave has an amplitude A at Pn, its amplitude is rA when it reaches P(n+1). Then, the shear wave that started out at time t1 at switching position P1 is amplified by a factor Aₙ=(1−r⁻⁽ⁿ⁺¹⁾)/(1−r) at a n^{th} switching position of focal region 42. The amplification tends asymptotically to a value A_{∞}=1/(1−r). Assume that a shear wave in tissue region 100 attenuates with an attenuation coefficient α per wavelength λ_{S} of the distance traveled by the shear wave, and that the distance between switching positions Pn and P(n+1) is equal to nλ_{S}. Then r=e−^{(αnλ}S⁾ and asymptotic value A_{∞} may be written A_{∞}=1/(1−e−^{(αnλ}S⁾). By way of numerical examples, if r is equal to 0.75, A_{∞}=4. If r is equal to 0.5, A_{∞}=2.

It is noted that resonant amplification of shear waves in accordance with an embodiment of the invention, such as illustrated in FIG. 2A, is not limited to ultrasound intensity configurations in which focal regions are rotated. In some embodiments, resonant amplification of a shear wave is also provided by lateral translation of a focal region with a velocity of the shear wave so that the radiation force is in phase with the shear wave. FIG. 2A, which is used to schematically represent resonant shear wave amplification by focal region 42 rotating along a circular trajectory, also represents resonant shear wave amplification for a focal region moving along any trajectory substantially laterally in the focal plane.

For example, a longitudinal ultrasound focal region created by longitudinal ultrasound of a frequency f_{L} has a lateral extent W˜λ_{L} (equal essentially to the ellipsoid minor axis). The radiation force produced at the focal region has a similar lateral extent and decreases from a maximum substantially in the middle of the focal region to zero at a radial distance λ_{L}/2 from the middle. The radiation force has a lateral extent equal to about ½ of the wavelength λ_{S} of the shear wave that it generates. Therefore, if the frequency of the shear wave is f_{S}, to provide resonant amplification of the shear wave, the focal region should be moved in the direction of propagation of the shear wave with a speed V_{S}(f_{S})=2Wf_{S}. If, for example, the focal region is generated by exciting an ultrasound transducer with two frequencies f₁ and f₂, so that the frequency f_{S} of the shear wave is equal to a beat frequency v=|f₁−f₂| between the excitation frequencies, then the condition for resonant amplification becomes V_{S}(v)=2Wv.

FIG. 2B shows schematically an ultrasound transducer 220 configured to generate and move a longitudinal ultrasound focal region along a linear trajectory so that it generates and resonantly amplifies shear waves, in accordance with an embodiment of the invention. Transducer 220 is optionally identical to transducer 20 shown in FIGS. 1A-1B but is excited differently than by rotating halves of the transducer that are excited with a phase difference of 180° about axis 28. Transducer 220 is excited by applying an AC voltage at a first frequency f₁, optionally equal to 3.0 MHz, to a first (shown shaded) half 231 of the transducer, and a second AC voltage at a frequency f₂, optionally equal 3.001 MHz, to a second opposite half 232 of the transducer. The excitation generates a focal region 222 in focal zone 100 and a corresponding radiation force 223 in the focal region that has a lateral extent W equal to about 0.5 mm. Note that W represents essentially the minor axis of the ellipsoid representing focal region 222. The focal region repeatedly traverses a linear path indicated by a block arrow 225 from one side to the other of focal zone 100 at a velocity equal to about 0.5 mm×1 kHz=0.5 m/s. Radiation force 223 generates shear waves, schematically represented by dashed circles 226, having a frequency equal to the beat frequency v=|f₁−f₂|=1 kHz and a corresponding velocity of propagation (at 1 kHz frequency) equal to about 0.52 m/s. The shear wave velocity is equal to the translation velocity of focal region 222, and radiation force 223 resonantly amplifies shear waves 226 as described above.

It is noted that focal region 222 can relatively easily be made to traverse linear trajectory 225 at a velocity substantially faster than 0.5 m/s and substantially faster than the velocity of shear waves that it generates. For example if v=|f₁−f₂|=10 kHz, the focal region moves with a velocity of 50 m/s and shear waves 226 propagate with a velocity of 1.64 m/s. For these velocities, the focal region moves “supersonically” with a Mach number equal to 50/1.64=30.5 and generates a shear shock wavefront. This, embodiments of the invention provide supersonic shear waves.

Shear wave generation and resonant amplification by rotating focal regions or translating focal regions, in accordance with an embodiment of the invention, provides shear waves having substantially enhanced intensity. The intense shear waves can be advantageous not only for imaging tissue, but also for delivering and coupling acoustic energy to a region of tissue to perform a diagnostic and/or therapeutic and/or cosmetic procedure on a patient's tissue. For example, for same intensity acoustic waves, tissue is generally more susceptible to disruption and/or destruction by acoustic shear waves than by ultrasonic longitudinal waves. As a result, for many types of procedures such as lithotripsy, tissue ablation and lysis, shear waves in accordance with an embodiment of the invention can be advantageous.

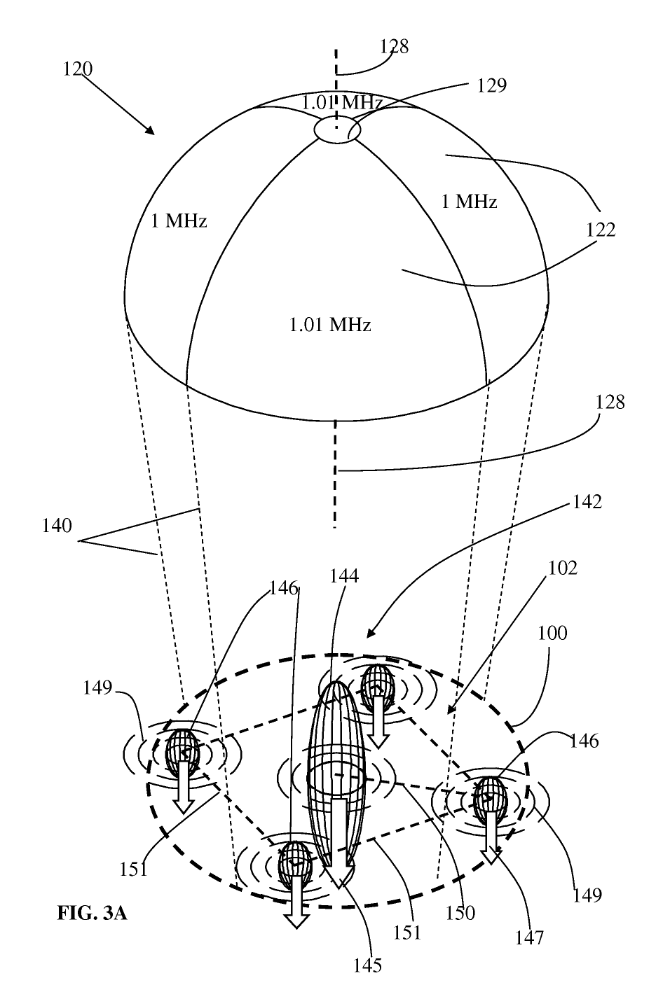

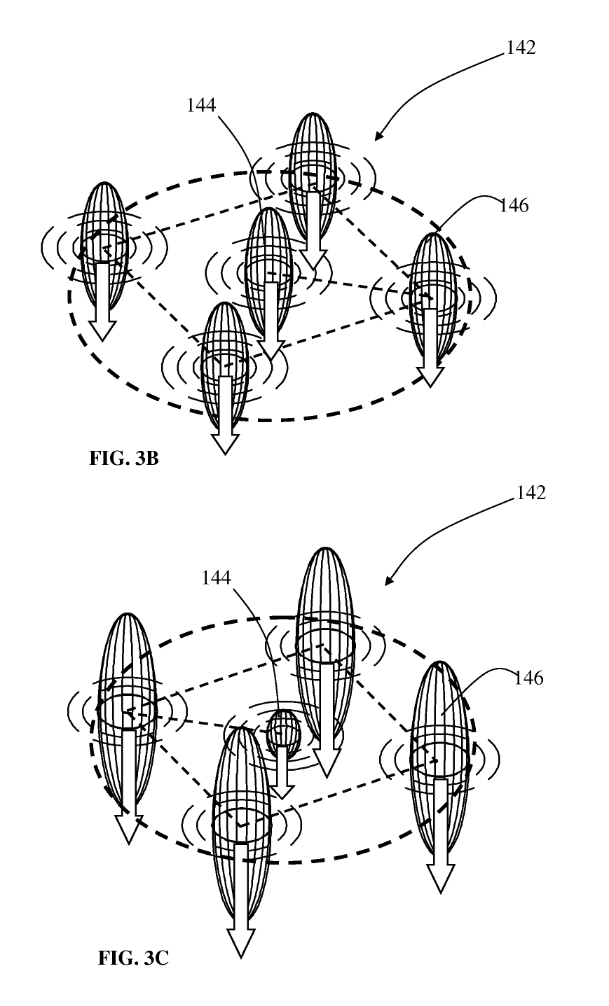

FIG. 3A schematically shows a multi-element spherical cap acoustic transducer 120 excited to generate a cyclically changing pattern of focal regions in a focal zone 102 of a tissue region 100 that provides amplified shear waves in the tissue region, in accordance with an embodiment of the invention. Cap transducer 120 is similar to cap transducer 20 and has a central axis 128 and a hole 129, but optionally four identical sectors 122. Each sector 122 has a sector electrode (not shown) on a convex surface 124 of the transducer for exciting the sector by applying a varying voltage between the sector's electrode and a common electrode on a concave surface 126 of the cap transducer. While shown as having specifically 4 sectors, it is to be understood that in general, the transducer can have any even number N of sectors or elements.

In accordance with an embodiment of the invention, sectors 122 in a first pair of opposite sectors are excited in phase with a first signal at a first frequency “f₁” to radiate longitudinal ultrasound at the first frequency to focal zone 102. Sectors 122 in a second pair of sectors are excited in phase with a second signal at a second frequency “f₂”, different from f₁, to radiate longitudinal ultrasound at the second frequency to the focal zone. More generally, odd numbered sectors of the N sectors can be excited in phase with signals at first frequency f₁ and even numbered sectors of the N sectors can be excited in phase with signals at second frequency f₂, different from f₁. Optionally, the first and second signals are continuously applied to their respective associated pairs of sectors. Dashed lines 140 schematically represent ultrasound radiated by sectors 122 to focal zone 102.

Radiated ultrasound 140 generates a pattern 142 of ultrasound intensity focal regions which comprises a “central” focal region 144 and four “peripheral” focal regions 146. In the general case of a transducer with N sectors, the pattern will have one central and N peripheral focal regions. The distance between central focal region 144 and each of peripheral regions 146 is represented by a dashed line 150. Adjacent peripheral focal regions 146 are separated by a distance represented by a dashed line 151. Pattern 142 repeatedly morphs through a cycle in which the intensity of ultrasound in central focal region 144 increases and decreases, while the intensity of ultrasound in peripheral focal regions 146 respectively decreases and increases. The cycle repeats at a frequency “v” equal to a difference between the first and second frequencies f₁ and f₂.

In FIG. 3A, focal region pattern 142 is shown at a first time of its cycle, in which intensity of ultrasound 140 radiated by cap transducer 120 in central focal region 144 is near a maximum, and intensity of ultrasound in peripheral focal regions 146 is near a minimum. The focused longitudinal ultrasound in central focal region 144 generates a radiation force 145 in the central focal region. The focused longitudinal ultrasound in peripheral focal regions 146 generates radiation forces schematically represented by block arrows 147. Because the cycle of increase and decrease of longitudinal ultrasound intensity in central focal region 144 is 180° out-of-phase with the increase and decrease of longitudinal ultrasound intensity in peripheral focal regions 146, radiation force 145 is 180° out-of-phase with radiation forces 147. Radiation forces 145 and 147 excite shear waves in each of the focal regions that radiate out from the region. These shear waves are represented by arcs 149.

FIG. 3B schematically shows focal region pattern 142 and radiation forces 147 at a second time during its cycle, at which time the intensity of ultrasound in central focal region 144 has decreased and the intensity of ultrasound in peripheral focal regions 146 has increased so that intensity in all the focal regions is about the same. FIG. 3C schematically shows intensity pattern 142 and radiation forces at a third time in the cycle, when the ultrasound intensity in central focal region 144 is near a minimum and the ultrasound intensity in each peripheral focal region 146 is near a maximum.

In accordance with an embodiment of the invention, the geometry of cap transducer 120, the excitation frequencies of sectors 122, and thereby the frequency of ultrasound 140 radiated to focal zone 102 to produce pattern 142 are determined so that shear waves 149 generated by radiation force 147 interfere constructively and are amplified resonantly. When pattern 142 is configured to provide resonant shear wave amplification, the pattern, as noted above, may be referred to as a “virtual resonator” and the resonance it provides may be referred to as “virtual resonance”.

The geometry of cap transducer 120 and frequencies f₁ and f₂ determine distances 150 and 151. The frequency f_{S} of shear waves 149 generated at each focal region is equal to v=|f₁−f₂|. Since radiation force 145 is 180° out-of-phase with radiation forces 147, to provide resonant amplification between shear waves 149 generated at focal region 144 and peripheral focal regions 146, distance 150 is advantageously equal to (n+½)λ_{S} where n is an integer and λ_{S} is the wavelength of shear waves 149 in tissue 100 for frequency f_{S}=v. For resonant amplification between shear waves 149 generated at peripheral focal regions 146, distance 151 or twice distance 150 is advantageously equal to nλ_{S} since radiation forces 147 are in phase. Note that for the abovementioned resonant amplification conditions, conditions for resonant amplification between shear waves 149 produced at the peripheral focal regions is generally not obtained simultaneously with conditions for resonant amplification between shear waves produced at central focal region 144 and peripheral regions 146.

By way of a numerical example of generation and resonant amplification of shear waves 149, assume that cap transducer 120 has an aperture equal to 85 mm and a radius of curvature equal to about 54 mm, and that frequencies of excitation f₁ and f₂, as shown in FIG. 3A, are respectively equal to 1.0 MHz and 0.99 MHz. As a result, frequency f_{S} of shear waves 149 is equal to 10 kHz, the velocity of the shear waves V_{S}(v) Unfortunately, the velocity of shear waves depends on their frequency (dispersion) is equal to about 1.64 m/s and their wavelength λ_{S} is equal to about 0.164 mm. Distance 151 is equal to about 1.5 mm, which is equal to 9λs, and resonant amplification results between shear waves 149 produced at peripheral focal regions 146.

By way of another numerical example, if f₁ and f₂ are equal to 1.0 MHz and 0.995 MHz, f_{S} is equal to 5 kHz, V_{S}(v) is equal to about 1.16 m/s and λ_{S} is equal to about 0.23 mm. Distance 151 is still equal to about 1.5 mm and distance 150 is equal to 1.05 mm, i.e. to about 4.5 λ_{S} As a result, there is resonant amplification between shear waves produced at central focal region 144 and shear waves produced at peripheral focal regions 146.

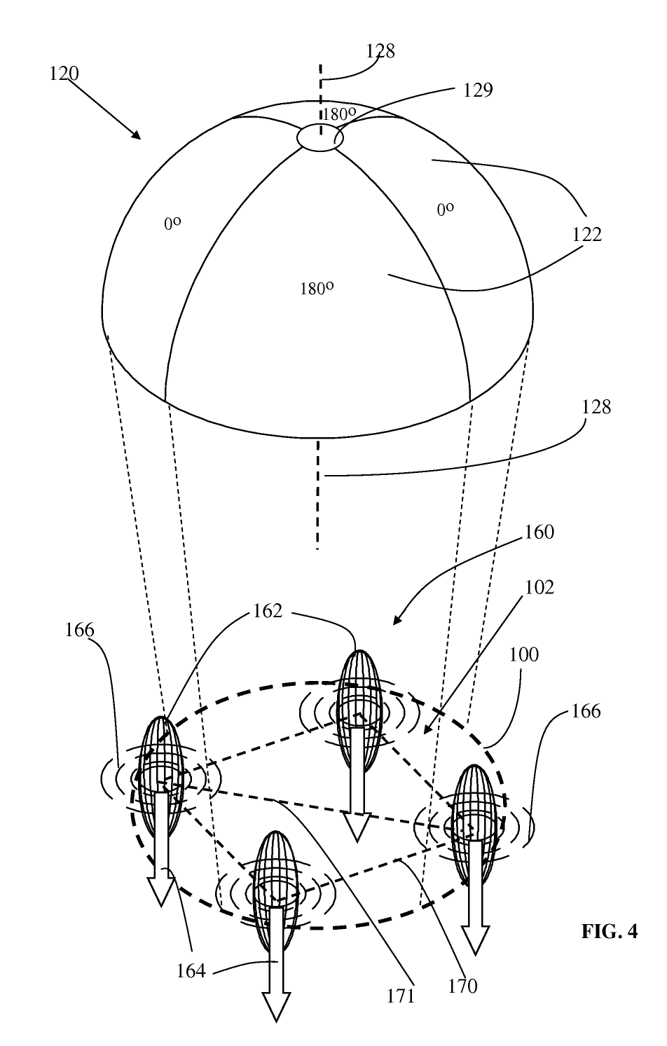

FIG. 4 schematically shows ultrasound cap transducer 120 shown in FIG. 3A excited to generate another longitudinal focal region pattern that provides resonantly amplified shear waves in tissue region 100, in accordance with an embodiment of the invention. While shown as having specifically 4 sectors, it is to be understood that in general, the transducer can have any even number N of sectors or elements. In FIG. 4, sectors 122 in different pairs of opposite sectors are simultaneously excited by bursts of a same frequency voltage, but sectors 122 in different pairs, as shown in the figure, are excited 180° degrees out-of-phase with respect to each other. The excitation provides a focal region pattern 160 comprising four longitudinal ultrasound focal regions 162 and associated radiation forces 164 in focal zone 102 which are symmetrically positioned with respect to axis 128. The radiation forces generate shear waves 166 that radiate away from each focal region 162. Focal regions 162 adjacent to each other are separated by a distance 170. Diagonally opposite focal regions 162 are separated by a “diagonal” distance 171. Focal region pattern 160 is stationary over time in the sense that the focal zones upon each burst appear in the same positions. Note that the focal zones appear as “pulsating’ zones.

In accordance with an embodiment of the invention, the geometry of cap transducer 120, the frequency of the excitation voltage and the burst repetition frequency are determined so that conditions for resonant amplification of shear waves 166 are obtained. Resonant amplification is obtained for distances 170 equal to nλ_{S} or for diagonal distance 171 equal to nλ_{S}.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

All patents, patent applications and publications mentioned in this specification are herein incorporated in their entirety by reference into the specification, to the same extent as if each individual patent, patent application or publication was specifically and individually indicated to be incorporated herein by reference. In addition, citation or identification of any reference in this application shall not be construed as an admission that such reference is available as prior art.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims

1. Apparatus for generating resonantly amplified ultrasound shear waves in biological tissue, comprising:
a) a plurality of transducer elements independently excitable to radiate ultrasound energy to generate a pattern of focal regions, each focal region having associated therewith a radiation force that generates a respective shear wave; and
b) a controller configured to control the element excitation such as to generate a pattern of focal regions which appear cyclically at different switching positions and to create resonant amplification of shear waves.

2. The apparatus of claim 1, wherein the transducer elements have substantially identical shapes and areas and are arranged symmetrically relative to a transducer symmetry axis.

3. The apparatus of claim 2, wherein the plurality of transducer elements includes a pair of first and second elements and wherein the element excitation includes applying bursts of AC voltage with a frequency f at a burst repetition frequency v, the bursts applied with one phase to the first element and with an opposite phase to the second element, the excitation resulting in two focal regions in a focal plane.

4. The apparatus of claim 3, wherein the two focal regions are separated by a distance equal to an integer number of shear wave wavelength nλ_{S}.

5. The apparatus of claim 4, wherein the pattern of the two focal regions in the focal plane pulsates at burst repetition frequency v.

6. The apparatus of claim 1, wherein the transducer elements are arranged in a rotationally symmetric configuration around a transducer symmetry axis.

7. The apparatus of claim 6, wherein the transducer elements include four identical elements and wherein the element excitation includes excitation with bursts of AC voltage with a frequency f at a burst repetition frequency v with one phase applied to a first pair of adjacent elements and with an opposite phase applied to a second pair of adjacent elements, the excitation resulting in two focal regions.

8. The apparatus of claim 7, wherein the excitation results in the two focal appearing cyclically at switching positions which are rotated around the transducer symmetry axis by 90° relative to a previous cycle.

9. The apparatus of claim 8, wherein the shear waves have a frequency equal to the burst repetition frequency v and wherein the excitation includes switching the phases applied to respective elements such that a first element in each pair switches to an opposite phase to the phase it had in an immediately previous cycle, the switching done with a switching time which equals a propagation time of the shear waves between the two focal regions, thereby causing the resonant amplification.

10. The apparatus of claim 8, wherein the shear waves have a frequency equal to the burst repetition frequency v and wherein the excitation includes switching the phases applied to respective elements such that a first element in each pair switches to an opposite phase to the phase it had in an immediately previous cycle, the switching done with a switching time shorter than a propagation time of the shear waves between the two focal regions to cause supersonic shear wave generation.

11. (canceled)

12. (canceled)

13. (canceled)

14. (canceled)

15. (canceled)

16. (canceled)

17. A method for generating resonantly amplified ultrasound shear waves in biological tissue comprising:
a) providing a plurality of transducer elements independently excitable to radiate ultrasound energy to generate a pattern of focal regions, each focal region having associated therewith a radiation force that generates a respective shear wave; and
b) exciting the transducer elements such as to generate a pattern of focal regions which appear cyclically at different switching positions and to create resonant amplification of the shear waves.

18. The method of claim 17, wherein the transducer elements have substantially identical shapes and areas and are arranged symmetrically relative to a transducer symmetry axis.

19. The method of claim 18, wherein the plurality of transducer elements includes a pair of first and second elements and wherein the step of exciting includes applying bursts of AC voltage with a frequency f at a burst repetition frequency v, the bursts applied with one phase to the first element and with an opposite phase to the second element, the excitation resulting in two focal regions in a focal plane.

20. The method of claim 19, wherein the two focal regions are separated by a distance equal to an integer number of shear wave wavelength nλ_{S}.

21. The method of claim 20, wherein the step of exciting includes generating a pulsating pattern of the two focal regions in the focal plane at burst repetition frequency v.

22. The method of claim 17, wherein the step of providing a plurality of transducer elements includes providing a plurality of transducer elements arranged in a rotationally symmetric configuration around a transducer symmetry axis.

23. The method of claim 22, wherein the transducer elements include four identical elements and wherein the step of exciting includes exciting the transducer elements with bursts of AC voltage with a frequency f at a burst repetition frequency v with one phase applied to a first pair of adjacent elements and with an opposite phase applied to a second pair of adjacent elements, the excitation resulting in two focal regions.

24. The method of claim 23, wherein the exciting further includes generating the two focal regions cyclically at switching positions which are rotated around the transducer symmetry axis by 90° relative to a previous cycle.

25. The method of claim 24, wherein the shear waves have a frequency equal to the burst repetition frequency v and wherein the step of exciting further includes switching the phases applied to respective elements such that a first element in each pair switches to an opposite phase to the phase it had in an immediately previous cycle, the switching done with a switching time which equals a propagation time of the shear waves between the two focal regions, thereby causing the resonant amplification.

26. The method of claim 24, wherein the shear waves have a frequency equal to the burst repetition frequency v and wherein the step of exciting further includes switching the phases applied to respective elements such that a first element in each pair switches to an opposite phase to the phase it had in an immediately previous cycle, the switching done with a switching time shorter than a propagation time of the shear waves between the two focal regions to cause supersonic shear wave generation.

27. (canceled)

28. (canceled)

29. (canceled)

30. (canceled)

31. (canceled)

32. (canceled)
